(19) United States
(12) Patent Application Publication
(10) Pub. No.: US 2009/0087380 A1
(43) Pub. Date: Apr. 2, 2009

(54) Polymer devices for therapeutic applications

(76) Inventors: Rainer J. Fasching, Mill Valley, CA (US); WonHyoung Ryu, Mountain View, CA (US)

Correspondence Address: LUMEN PATENT FIRM, 2345 YALE STREET, SECOND FLOOR, PALO ALTO, CA, 94306, US

(21) Appl. No.: 12/284,168

(22) Filed: Sep. 17, 2008

Related U.S. Application Data
(63) Continuation-in-part of application No. 11/402,651, filed on Apr. 11, 2006.
(60) Provisional application No. 60/670,483, filed on Apr. 11, 2005.
(60) Provisional application No. 60/994,216, filed on Sep. 17, 2007.

(51) Int. Cl. A61K 49/00; A61K 9/00; A61P 43/00; A61K 47/30

(52) U.S. Cl. 424/9.1; 424/400; 514/772.3; 424/422; 424/428

(57) ABSTRACT

Multi-layered polymer devices having three-dimensional containers for holding a therapeutic and/or imaging agent are provided. The devices have a high loading ratio of agent to polymer material for effective treatment. Delivery wings or regions with channels connected to the containers are also incorporated in the devices. The delivery wings can be flexible and insertable into hollow instruments, such as a needle. Anchoring structures are also provided for fixing the position of the device after injection into a subject. The polymer layers of the device can be biodegradable. Biodegradable materials can also be used to provide controlled release of agents, such as for immunizations and other therapeutic or non-therapeutic applications.

### CROSS-REFERENCE TO RELATED APPLICATIONS

This application claims priority from U.S. Provisional Patent Application 60/994,216 filed Sep. 17, 2007, which is incorporated herein by reference. This application is a continuation-in-part of U.S. patent application Ser. No. 11/402,651 filed Apr. 11, 2006, which claims priority from U.S. Provisional Patent Application 60/670,483, filed on Apr. 11, 2005, all of which are incorporated herein by reference.

### FIELD OF THE INVENTION

The invention relates generally to drug delivery. More particularly, the present invention relates to drug delivery devices with high loading ratios.

### BACKGROUND

Controlled delivery of therapy has been of great interest in medicine for many years, especially in cases where it is undesirable or impractical to provide frequent doses of therapy. For example, timed-release tablets or capsules of various kinds have been developed to reduce dosage frequency, release ingested drugs in specific parts of the digestive system, and other variations. Such tablets tend to rely on biodegradation of tablet materials to provide a more controlled release of drugs than would otherwise be obtained.

Another approach for providing controlled therapy is a device having multiple reservoirs of an agent to be delivered. For example, US 2004/0248320 considers such a device where each reservoir is individually electrically controllable such that a reservoir cap can be selectively disintegrated or permeabilized, thus releasing the agent. U.S. Pat. No. 6,010,492 and US 2006/0057737 also consider devices having reservoirs, which can be independently actuated to control drug release. A passive device having a drug reservoir is considered in US 2005/0118229, where release is controlled by a composite nano-porous/micro-porous membrane covering the reservoir.

Controlled therapy by providing polymer multi-layers including a drug-loaded layer has also been considered, e.g., as in U.S. Pat. No. 6,322,815, U.S. Pat. No. 5,603,961, and U.S. Pat. No. 6,316,018. Such polymer multi-layers often include one or more porous layers. Porous layers can be loaded with one or more drugs in the pores and/or can be used to control the drug delivery rate. Multi-layer drug-releasing constructs have found various applications, including vascular graft and stent covers (U.S. Pat. No. 6,702,849), drug delivery via a patch applied to mucosal tissue (US 2003/0219479), and transdermal drug delivery (U.S. Pat. No. 5,273,756 and U.S. Pat. No. 3,797,494).

Although it is clear that controlled drug delivery has been extensively investigated, not all issues have been completely resolved. For example, in cases where a drug is incorporated into a degradable structure to control delivery, it is necessary to ensure that the degradation products of the structure do not interfere with the drug being delivered. Furthermore, it can be difficult to control the drug release rate by controlling the degradation process. In cases where a porous polymer layer is used to hold drugs and/or to control the delivery rate, the delivery rate can depend sensitively on parameters of the porous layer (e.g., porosity, mean pore size, degradation rate), which are imperfectly controlled during fabrication. For example, two membranes made in different ways (or by different manufacturers) may have different drug delivery properties even if they nominally have the same pore size and porosity.

Another issue with miniaturized, implantable, oral, or other types of drug delivery devices is the amount of material needed to construct the device relative to the amount of agent that can be loaded into the device. Loading an amount of biological or non-biological agent into a structure is often limited by the thickness of the device material. Often, there is a very low ratio of agent to material. With limited agents, the desired therapy can have a reduced effectiveness. Furthermore, the large amount of materials required in existing device (to provide an adequate amount of agent) can cause harmful effects to a subject.

The present invention addresses at least these difficult problems and advances the art with an agent delivery device capable of having a high loading ratio.

### SUMMARY OF THE INVENTION

The present invention is directed to a device for releasing an agent to subject. The device includes a first polymer layer and a second polymer layer, wherein the polymer layers are partially bonded to form a bonded region and a non-bonded region. The non-bonded region forms at least one container located between the first and second polymer layers, wherein the container holds the agent to be released to the subject. The bonded region includes a delivery region, such as a delivery wing, comprising at least one channel located between the first and second polymer layers. The channel connects the container to an external environment of the device. In a preferred embodiment, the device has a high loading ratio of the agent to the polymer material of the polymer layers, wherein the high loading ratio is greater than about 0.5. In another embodiment, the container has a thickness ranging from about 0.01 mm to about 3 mm.

The polymer layers can be biodegradable and/or biocompatible, and the agent can be a therapeutic agent for providing therapy to the subject. In an embodiment, the polymer layers have an in vivo lifetime greater than the duration of the therapy. The device can also include a biodegradable material that at least partially blocks the channel for delayed release of the agent.

In a preferred embodiment, the delivery wing is flexible, and can be folded or rolled to fit inside a hollow instrument, such as a needle, a catheter, an endoscope, or a syringe. In certain embodiments the flexible delivery wing unfolds after insertion into a subject.

The device can include any number of containers and channels. In a preferred embodiment, the device includes multiple containers with a channel corresponding to each container for connecting the same container to the external environment of the device. The multiple containers can hold the same or different agents to be released. At least two of the channels can have different geometries to provide different delivery rates of agents from their corresponding containers. In another embodiment, a first biodegradable material at least partially blocks a first channel and a second biodegradable material at least partially blocks a second channel. The first and second biodegradable materials can have different in vivo lifetimes. Devices with multiple containers can also include one or more mixing channels for connecting two or more containers.

In embodiments of the present invention, the device has a sheet structure for releasing the agent over a large area. Alternatively, the device can have a tubular shape or disc shape.

In embodiments, the device includes an anchor, such as a jagged structure, a suture opening, a hook, a needle, a pin, a hole, a slit, a knob, or any combination thereof, for maintaining a position of the device. In an embodiment, the device with anchor is implantable in an eye of a subject.

In radiation therapy guidance device is also provided. The guidance device includes a first polymer layer and a second polymer layer, wherein the first and the second polymer layers are partially bonded to form a bonded region and a non-bonded region. The polymer layers are preferably biodegradable or biocompatible. The non-bonded region forms at least one container located between the first and the second polymer layers. A radiation therapy imaging marker can be stored inside of the container or attached on the outside of the container. The imaging marker can be one or more solid particles, wherein the solid particles include metal, metal alloy, gold, carbon, or any combination thereof. Additionally or alternatively, the imaging marker can include a metal film, wherein the metal film is coated on the radiation therapy guidance device. In a preferred embodiment, the guidance device includes an anchor for maintaining the position of the device.

In certain embodiments, a radiation therapy guidance device also includes at least one channel connecting the container to the external environment of the device. The container can hold agents to be released through the channel.

### BRIEF DESCRIPTION OF THE FIGURES

The present invention together with its objectives and advantages will be understood by reading the following description in conjunction with the drawings, in which:

FIGS. 1A-B show an example device having a container and delivery wings with channels between two polymer layers and a cross-sectional view of the device according to the present invention.

FIGS. 2A-C show example devices for releasing an agent, some of which include multiple containers and channels with different geometries according to the present invention.

FIGS. 3A-B show example devices having multiple connected containers according to the present invention.

FIG. 4 shows an example device having a container and channels at the ends of the container according to the present invention.

FIGS. 5A-B show an example device having a single delivery wing and a cross-sectional view of the device according to the present invention.

FIGS. 6A-C show example devices with flexible delivery wings according to the present invention.

FIG. 7 shows an example device having a sheet structure with multiple containers and channels according to the present invention.

FIG. 8 shows an example device having a tubular shape with multiple containers and channels according to the present invention.

FIG. 9 shows an example device having a mesh sheet structure according to the present invention.

FIG. 10 shows an example device with blocked channels for delayed release of agents according to the present invention.

FIG. 11 shows an example device having anchoring structures according to the present invention.

FIGS. 12A-B show an example of a radiation therapy guidance device and a cross-sectional view of the device according to the present invention.

FIGS. 13A-B show an example of a radiation therapy guidance device with channels and a cross-sectional view of the device according to the present invention.

FIGS. 14A-C shows an example method for fabricating a device according to the present invention.

FIGS. 15A-C show an example method for fabricating a device using two molded polymer layers according to the present invention.

FIG. 16 shows an example large sheet fabrication method using rolling process for molding and bonding polymer layers according to the present invention.

FIG. 17A shows an example of a prior art aqueous or gel depot for sclera treatment.

FIG. 17B shows an example device for treatment of the eye with approximately unidirectional drug flow according to the present invention.

FIGS. 18A-B show an example device having sealing rings and a cross-sectional view of the device according to the present invention.

FIG. 19 shows a plot of drug release versus time from in vivo experimental studies using a device of the present invention compared with in vitro results.

### DETAILED DESCRIPTION OF THE INVENTION

A device having a multi-layered polymer structure with a high loading ratio of agents or biological substances to polymer material is described herein. In certain embodiments, the loading ratio can be greater than about 0.5, up to about 0.9, or higher. For a controlled release or activation of the loaded substances, channels and/or conduits can be incorporated in these structures. The structures can allow for fabrication of tubular shaped devices that can be administered with hollowed instruments such as small needles. Foldable and/or rollable sheet devices and three-dimensional shaped devices for placement in voids and tubes are described as well. Further, the device can be equipped with anchoring structures to avoid migration and movement of the device from the site where it was placed, for example in biological tissues.

A fabrication scheme is also provided to offer the possibility of loading an agent after the devices are constructed. Particularly, post-loading of an agent offers high flexibility in agent incorporation and enables incorporation of sensitive agents in respect to temperature, solvent, or mechanical forces such as big molecules or reactive substances.

Also, guidance devices are provided. The guidance devices herein allow for loading of active or inactive agents at high enough density (due to the high loading ratio) that is detectable with standard imaging methods (e.g., radiological methods such as X-ray imaging, CT, and MRI), enabling the tracing and localization of the devices after administration or implantation. A coding system can be generated by patterning of the incorporated active agents and enables differentiation between multiple structures. These structures can be used to guide therapeutic radiation for local therapies.

The devices may be fabricated from biodegradable or non-biodegradable materials. Devices may be used as implantable devices for a large variety of therapeutic and non-therapeutic applications including, but not limited to, drug delivery, combinational drug delivery, image guidance for local therapies, radiation guidance for local therapies, cell-based drug delivery, tissue engineering, and immunizations. The device may be used for non-medical applications as well.

One embodiment of the invention is a two-layer polymer structure comprising a bonded area and a non-bonded area. The non-bonded area forms a geometrical three-dimensional container unit (e.g., cylinder, cube, cone). The container provides a volume for loading an agent. The volume of the structure defines the amount of required polymer. The container is loaded with a therapeutic or non-therapeutic agent from the group consisting of: a liquid, gas, paste, powder, or solid. In the case of therapeutic agents, loading ratios are typically of clinical importance. Loading of agents can be performed in various ways (e.g., micro dispensing, micro injection, powder compaction, screen-printing, ink jet printing, or sieving). For agents in liquid form, loading can rely on capillary action. Loading of agents into the structure can be performed before or after fabrication of the multi-layered structure. In the device discussed herein, the ratio of drug to polymer increases proportionally with the volume of three-dimensional container and allows for loading ratios (agent to layer material) of greater than about 0.5 or even up to and greater than about 0.9.

In embodiments of the invention meant for delivery of a therapeutic agent, structures on the micro or nano scale are incorporated into the bonded area to control the release of the agent loaded within the containers. Small-scale conduits control agent delivery through diffusive or osmotic properties and release agents loaded within the container from the device to an external environment. Further details of agent delivery through diffusive or osmotic properties can be found in U.S. patent application Ser. No. 11/402,651 filed Apr. 11, 2006, which is incorporated herein by reference in its entirety.

Examples of conduits are, but are not limited to, channels, holes, pores, or a combination thereof. Areas adjacent to the container comprising a conduit are referred to herein as delivery regions. The delivery regions can be located on a side, at the top, or at the end of the container and can have any size or geometry. The delivery regions can be embedded in the bonded and/or the non-bonded areas during fabrication of the device or are fabricated in a separate procedure. In some embodiments, the delivery regions include the bonded region of the device. The delivery regions can include delivery wings comprised of flexible or non-flexible polymers. Flexible delivery wings allow the device to fold or roll. A folded or rolled device can then be placed into a hollow instrument, such as a needle or endoscope. In other words, a folded or rolled embodiment of the invention herein is injectable. The design of the device is amenable to many different variations including, but not limited to, those detailed in this description and the corresponding figures.

### Polymers

In a preferred embodiment, the device includes a first and a second polymer layer. The first and the second polymer layers can comprise the same or different polymers from the group consisting of: biocompatible polymers, biodegradable polymers, non-biodegradable polymers, water soluble polymers, and water insoluble polymers. In certain embodiments of therapeutic devices with biodegradable polymer layers, the biodegradable polymer layers have an in vivo lifetime greater than the duration of the therapy. In other words, the polymer layers will degrade after some or all of the agent has been released from the device.

Suitable biodegradable polymers include, but are not limited to: aliphatic polyesters, poly(amino acids), copoly(ether-esters), polyalkylene oxalates, polyamides, poly(iminocarbonates), polyorthoesters, polyoxaesters, polyamidoesters, polyoxaesters containing amine groups, poly(anhydrides), polyphosphazenes, polyoxaamides and polyoxaesters containing amines and/or amino groups, and blends thereof. Polyanhydrides from diacids of the form HOOC—C6H4-O—(CH2)mO—C6H4-COOH where m is an integer in the range of 2 to 8 and copolymers thereof with aliphatic alpha-omega diacids of up to 12 carbons are also suitable.

Aliphatic polyesters include but are not limited to homopolymers and copolymers of lactide (which includes lactic acid, d-, l- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone, hydroxybutyrate (repeating units), hydroxyvalerate (repeating units), 1,4-dioxepan-2-one (including its dimer 1,5,8,12-tetraoxacyclotetradecan 7,14-dione), 1,5-dioxepan-2-one, 6,6-dimethyl-1,4-dioxan-2-one 2,5-diketomorpholine, pivalolactone, alpha,alpha diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof.

Suitable non-absorbable polymers include but are not limited to: poly(dimethylsiloxane), silicone elastomers, polyurethane, poly(tetrafluoroethylene), polyethylene, polysulfone, poly(methyl methacrylate), poly(2-hydroxyethyl methacrylate), polyacrylonitrile, polyamides, polypropylene, poly(vinyl chloride), poly(ethylene-co-(vinyl acetate)), polystyrene, poly(vinyl pyrrolidine).

Suitable water soluble polymers include but are not limited to: saccharides such as cellulose, chitin, dextran, proteins such as collagen and albumin, acrylates and acrylamides such as poly(acryl acid), polyacrylamide, and poly(1-hydroxyethyl methacrylate), and poly(ethylene glycol).

Suitable water insoluble polymers (and other layer materials) include but are not limited to: yellow wax, petrolatum cholesterol, stearyl alcohol, white wax, white petrolatum, methylparaben, propylparaben, sodium lauryl sulfate, propylene glycol, glycerogelatins, geling agents such as carbomer 934, cellulose derivatives, natural gums, penetration enhancers such as dimethyl sulfoxide, ethanol propylen glycol, glycerin, urea, glycerogelatins, coloring agents, lactose, stearic acid, starch glycolate, sugar, gelatin, fixed vegetable oils and fats, glycerin, propylene glycol, alcohol, ethyl oleate, isopropyl myristate, dimethyl acetamide, and mixtures or aqueous or oil based dispersions of these.

Other suitable materials usable for the device, particularly for the first and second polymer layers, are described in U.S. patent application Ser. No. 11/402,651 filed Apr. 11, 2006, which is incorporated herein by reference in its entirety.

### Container and Channel Structures

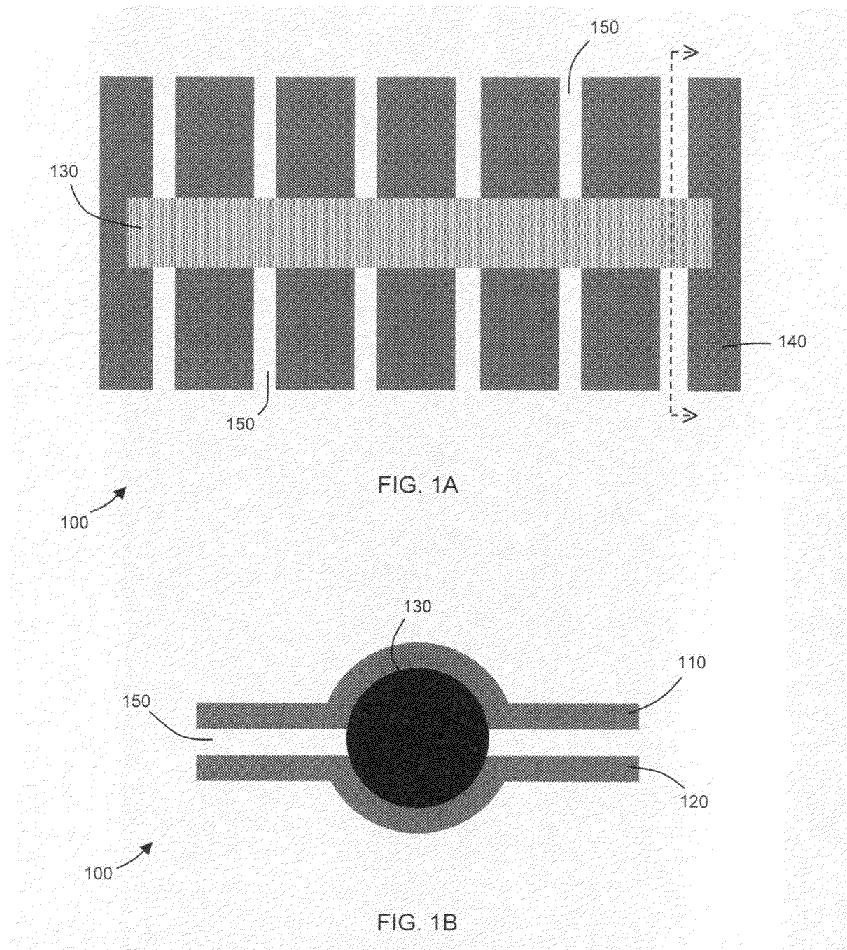

FIG. 1A shows an embodiment of a device 100 of the present invention and FIG. 1B shows a cross-sectional view of the device 100 along the dashed line shown in FIG. 1A. The device 100 includes a first polymer layer 110 partially bonded with a second polymer layer 120. The non-bonded region includes a container 130 for holding an agent to be released. The bonded region includes a delivery wing 140 and channels 150 incorporated into the delivery wing 140. The channels 150 connect the container 130 to an external environment of the device 100. It is important to note that the container 130 is three-dimensional and capable of holding a large amount of agent. In a preferred embodiment the container 130 has a thickness ranging from about 0.01 mm to about 3 mm or more preferably from about 0.2 mm to about 3 mm. For example, FIGS. 1A-B show a cylindrical container 130 having a circular cross section with a diameter ranging from about 0.2 mm to about 3 mm. In contrast to the container 130, the delivery wing 140 is an approximately two dimensional structure having a small thickness compared to the other two dimensions. This feature allows the device to have a high loading ratio.

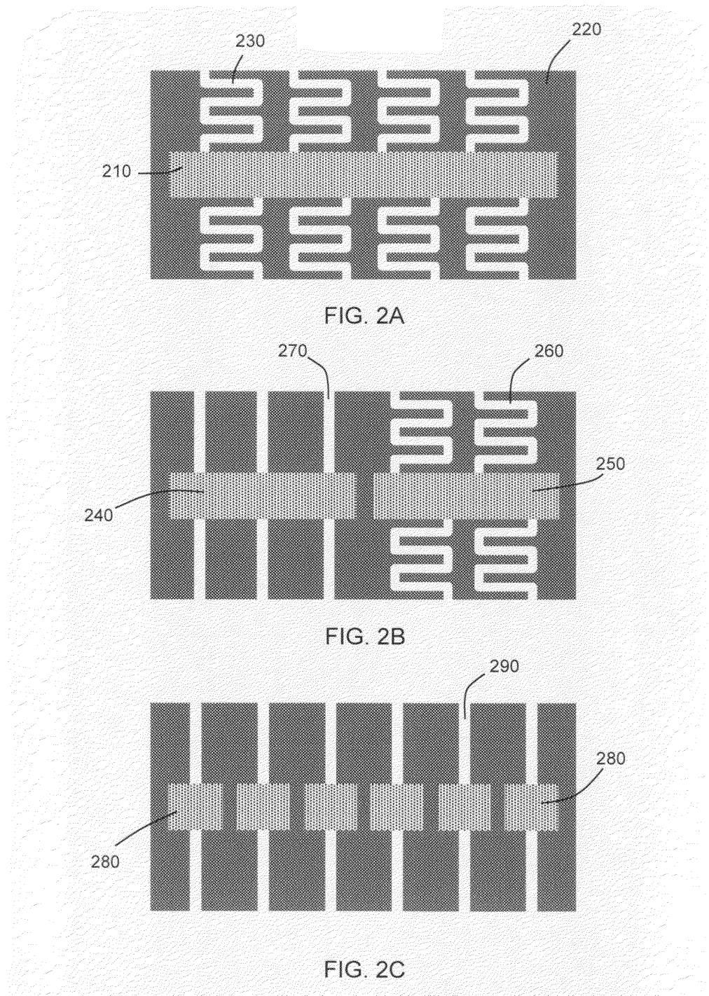

In the example shown in FIG. 1A, multiple straight channels 150 connect a single container 130 to an external environment of the device 100, demonstrating a device 100 that would be useful for delivering a large amount of a therapeutic agent and evenly dispersing the agent near the device 100. FIGS. 2-5 show alternative embodiments to the device 100 of FIG. 1A. In general, it will be appreciated by one of ordinary skill in the art that the device of the present invention can have any number and geometry of containers and channels.

FIG. 2A shows an embodiment of a device having a container 210 and a delivery wing 220 with channels 230. Unlike the straight channels 150 of device 100, channels 230 have a serpentine geometry to provide a longer pathway for the delivery of agents from the container to the environment. Channels can be shaped in any manner, whereby the shape, length, cross-sectional area, or geometry of the channel provides different delivery rates of the agent. By predetermining the area and length of a channel, modulated control of the release of an agent to the environment can be tailored for different types of agents and treatments by calculating the time and rate of release of the agents through diffusive or osmotic mechanisms.

FIG. 2B shows another embodiment of a device having multiple containers, a first 240 and a second 250 container, with multiple channels 260, 270 connected to the containers. Serpentine channels 260 are connected with container 250 and straight channels 270 are connected with container 240. The containers 240, 250 can be loaded with the same agent or different agents. As noted above, the different channel geometries can provide different delivery rates of agents held in containers 240, 250. For example, when both containers 240, 250 hold the same agent, and if channels 260, 270 have the same cross-sectional area, the shorter straight channels 270 deliver the agent in a shorter time than the longer serpentine channels 260. If both containers contain agents with different properties, such as density or viscosity, then the different length channels could be predetermined to deliver the different agents from the containers at the same time.

FIG. 2C shows an embodiment of an agent-delivery device having an array of containers 280, connected to the external environment through channels 290. The containers 280 in this example can also be connected to different length and different sized channels to control the time and rate of the release of agents through diffusive and osmotic mechanisms.

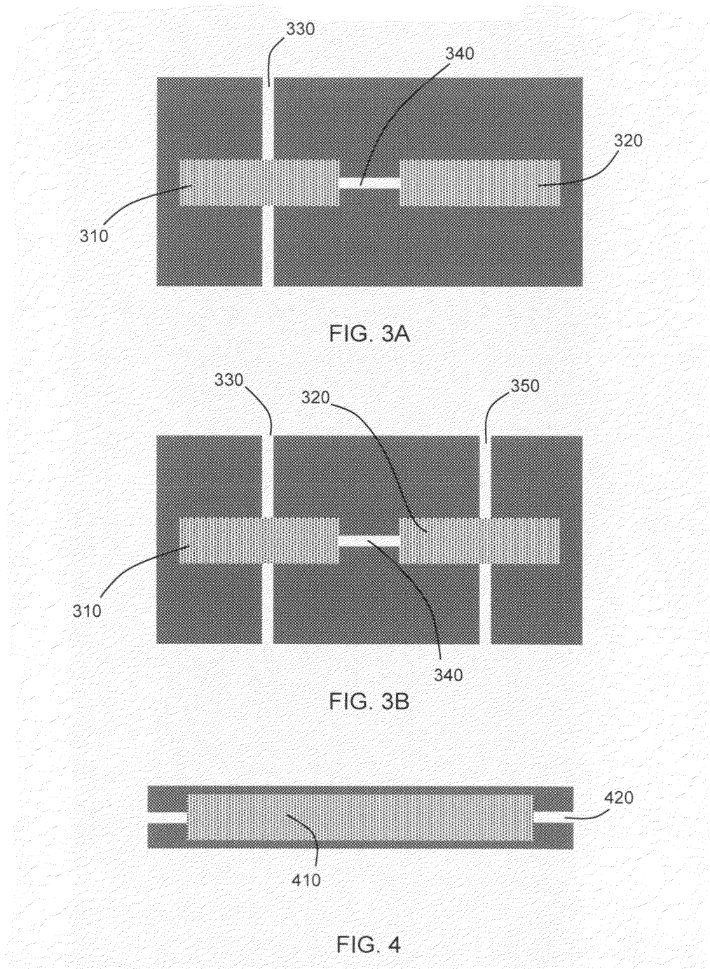

In certain embodiments of the present invention, a device includes a channel connecting multiple containers to control the movement of an agent from one container to another or provide a method for mixing agents loaded in separate containers. FIG. 3A shows a device wherein a first container 310 connects to a second container 320 through a mixing channel 340. The channel 340 in between containers controls the release of an agent or mixing of agents between containers. The device of FIG. 3A includes a channel 330 that connects container 310 to the external environment. Any number of containers of the device can be connected by a channel to the environment. For example, FIG. 3B shows a similar device as the one in FIG. 3A with the addition of a channel 350 for connecting container 320 to the environment.

The region where the agent exits the device is generally referred to as a delivery region. In some embodiments the channels are not necessarily incorporated into a delivery wing, and an agent can exit via a channel in any direction, including from the top of a container. In other words, the delivery regions may or may not include delivery wings. FIG. 4 provides an example of channels 420 exiting from the top of a container 410 and not through a delivery wing.

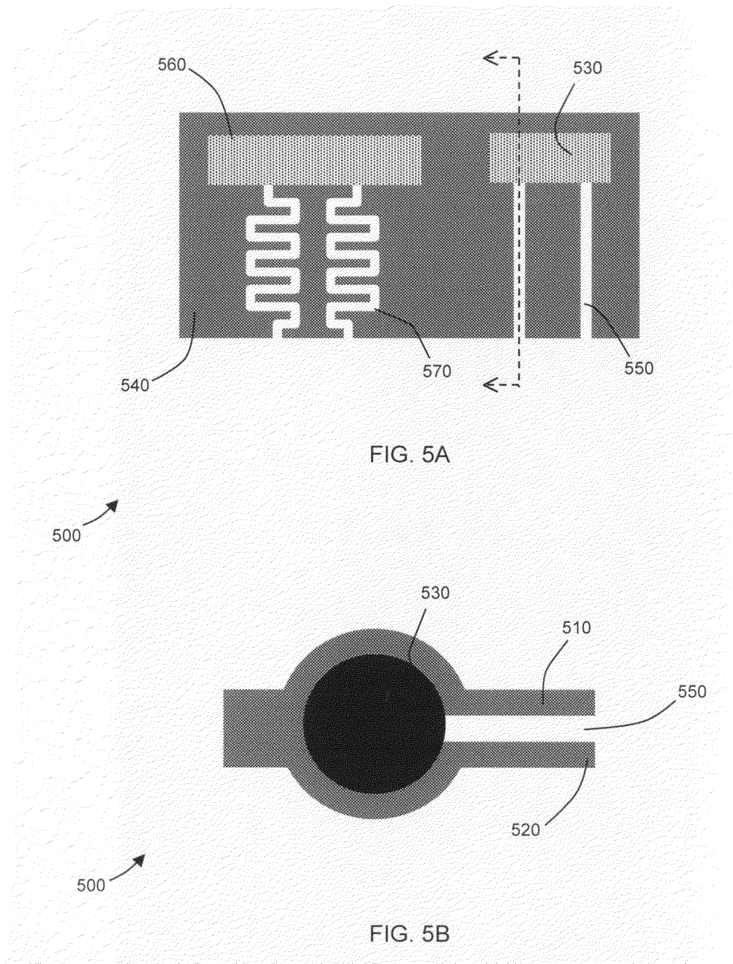

FIG. 5A shows an embodiment of the present invention that also includes a device 500 having a single delivery wing 540 to release an agent from one or more containers 530, 560. Device 500 includes two containers 530, 560 located to one edge of the device 500. FIG. 5B shows a cross-sectional view of the device 500 along the dashed lines shown in FIG. 5A. Device 500 includes a first polymer layer 510 partially bonded with a second polymer layer 520. Similar to devices with multiple delivery wings, device 500 can have any number of containers and channels, such as straight channels 550 connected to container 530 and serpentine channels 570 connected to container 560.

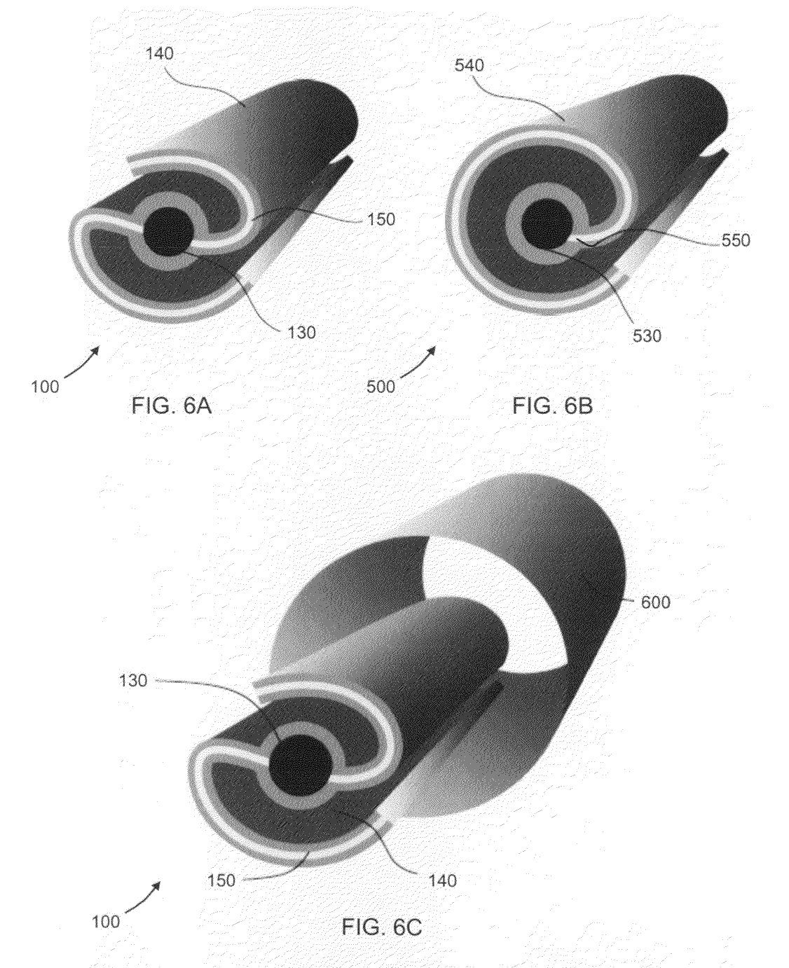

It is important to note that an embodiment of the present invention is directed to devices with one or more flexible delivery wings, examples of which are shown by FIG. 6. FIG. 6A shows a device 100 having multiple delivery wings that can be folded or rolled around a container 130. As described above, channels 150 can be positioned in the flexible delivery wings 140. FIG. 6B shows a device 500 having a single delivery wing 540 that can be rolled around a container 530. Similar to device 100, device 500 includes channels 550 in the delivery wing 540.

An embodiment of the invention involves inserting a folded or rolled device in a hollow instrument 600, as shown in FIG. 6C. The hollow instrument can be a needle, a catheter, an endoscope, a syringe, a hollow medical instrument, or any other hollow instrument capable of delivering said folded device to a subject. The folded or rolled device can be delivered to a target site by a simple injection procedure. During the administration process the delivery wings may unfold, helping to secure the device in place.

### Sheet Systems

In embodiments of the present invention, the expansion of the single unit or single device design to a multiple unit structure results in a sheet-shaped two-layer system. The shape and layout of the container system, as well as delivery region structures, can be changed and optimized for each application. An embodiment of this invention is a sheet with multiple containers, wherein each container is connected to one or more conduits to release an agent to the environment. The sheet structures can be folded or rolled before and/or during an administration procedure. The sheet shape allows for a uniform coverage of a large area. The two layer structure can be designed as a mesh with high fluid permeability or as a solid sheet in order to block fluid exchange between the independent sides of the device.

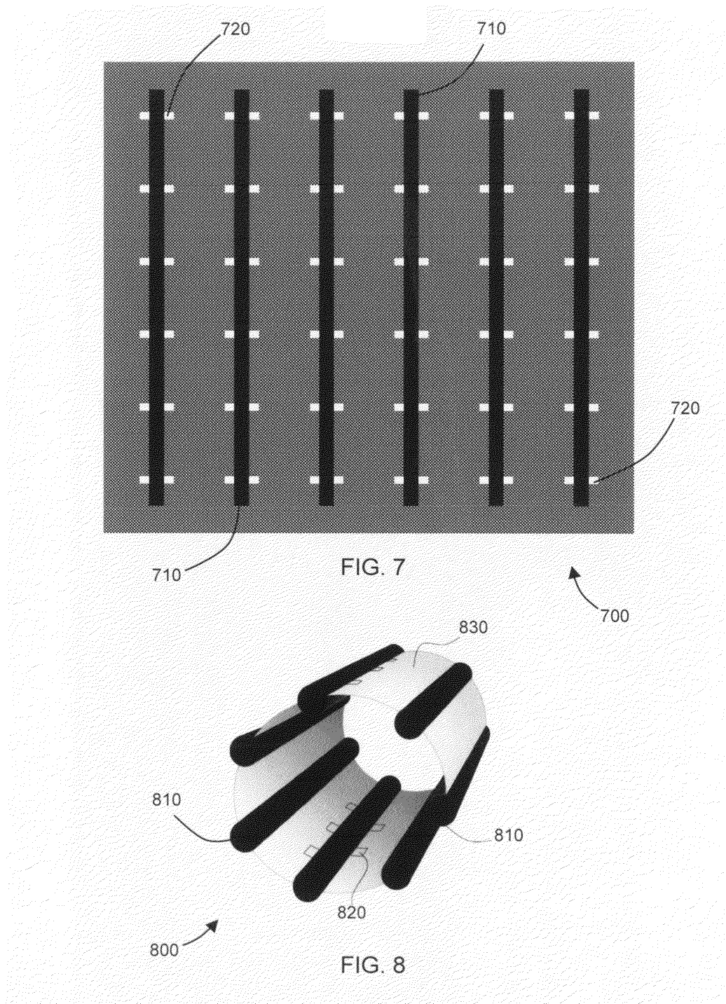

FIG. 7 shows an embodiment of a device 700 having multiple containers 710 arranged in a two dimensional sheet structure. The device 700 includes multiple conduits or channels 720 for releasing an agent to one side of a solid sheet and preventing the agent from dispersing on the other side of the sheet. The configuration can provide a large surface area on one side of the device 700 for therapy delivery. Alternatively, the channels 720 can deliver the agent to both sides of the device 700 or different agents to different sides of the device 700.

FIG. 8 shows another embodiment of the invention useful for stent- or tubular device-based therapy. The configuration is similar to the configuration in FIG. 7, wherein the device 800 comprises a solid sheet 830 with multiple containers 810 and multiple channels 820 connecting to each container 810. However, the solid sheet 830 is rolled into a tube. This tube configuration allows for the delivery of therapeutic agents to only the inside of the tube, to only the outside of the tube, or to both the inside and the outside of the tube.

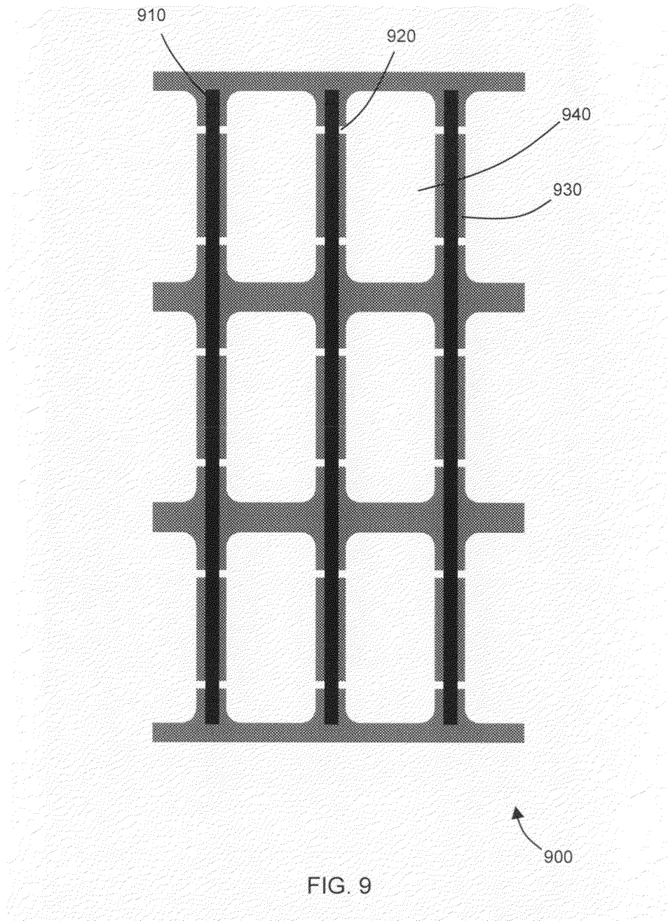

FIG. 9 shows yet another embodiment of a device 900 having a sheet configuration. Device 900 includes multiple containers 910 arranged on a mesh comprising a sheet 930 with voids 940 between bonded areas. Each container connects to one or more channels 920 to release an agent to the environment. The mesh allows the agent to more evenly disperse in the environment. The configuration can provide a large surface area for therapy delivery.

It is noted that the scope of the present invention also includes sheet structures of any shape, including shapes not displayed in the figures. For example, devices can be disc-shaped with concentric ring-shaped containers.

### Controlled Release

In a preferred embodiment, a feature of this invention involves controlling the timed release of agents into the environment. The channel size and shape can differ to offer different delivery properties. Another method of controlling release of an agent from a container to the environment is at least partially blocking a channel with a biodegradable material, such as a biodegradable polymer. When the polymer degrades, the agent releases to the environment according to the diffusive properties of the agent and conduit. In preferred embodiments, the polymer filling the channel degrades in a shorter time period than the polymer layers constructing the containers and delivery regions. For example, the in vivo lifetime of the biodegradable material blocking the channel is less than the in vivo lifetime of the biodegradable polymer layers. The same materials used for the polymer layers can be used to block a channel.

Blocking channels with a biodegradable material can be useful in treating diseases where long-term controlled release of a therapeutic agent is in demand. Unlike other embodiments of the invention that control the release of an agent through diffusive and osmotic properties, an embodiment with blocked channels can sustain longer time agent release by blocking the delivery channels with a biodegradable polymer. In a preferred embodiment, the polymer chosen to block the delivery channel would not be reactive with the agent within the container. The long-term controlled release is useful for delivering vaccines, immunizations, or therapy regimens requiring multiple doses spread out over a period of time, such as in any treatment requiring “booster” injections. One dose is released, followed by the release of another dose after a period of time predetermined by the conduit and the degradation time of the polymer chosen to block the delivery conduit. Depending on the number of doses needed, an embodiment of the invention comprises enough containers and delivery conduits blocked with successively degrading polymers to adequately deliver the treatment.

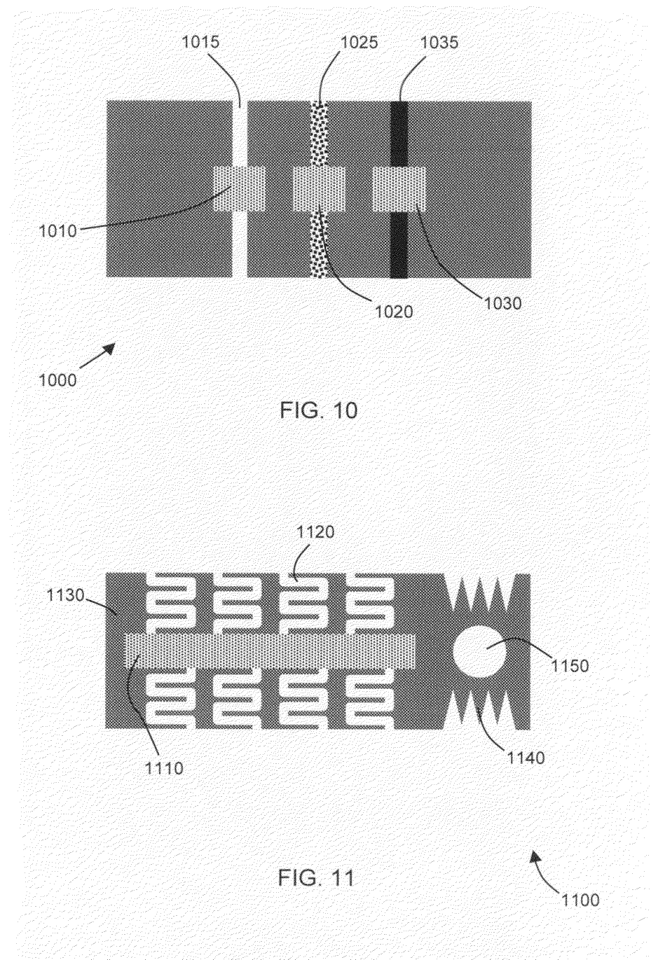

FIG. 10 shows an embodiment 1000 of the invention having multiple containers 1010, 1020, and 1030 connected to the external environment by channels 1015, 1025, and 1035, respectively. The channels 1015 connected to a first container 1010 are not blocked, the channels 1025 connected to a second container 1025 are blocked by a first blocking polymer, and the channels 1035 connected to a third container 1035 are blocked by a second blocking polymer. The first and second blocking polymers can have different degradation times. As would be appreciated by one of ordinary skill in the art, other configurations of blocking polymers and channels can also be implemented. For example, channels 1015 can also include a blocking polymer that can be the same or different than the blocking polymers in channels 1025 or channels 1035.

Immunization requires administration of an initial dosage and one or more follow up dosages of the vaccines at certain time periods. Immunization for multiple diseases therefore requires multiple injections distributed over a time period over half or one year. An embodiment of the invention comprises an implanted device that enables administration of all dosages for single and multiple immunizations with a single injection. The different dosages are loaded in individual device containers. Each container is connected with conduits to the outside. The delayed release is achieved by completely or partially filled channels with a material that will be dissolved and open the container to deliver the next dosage at a programmed time line.

Solvents or substances that are immiscible with water such as DMSO, Fluorocarbon solvents, or oil can be used to avoid exposure of a loaded agent to humidity or water, and therefore, avoid destabilization of the agent. The agent can be mixed or dissolved in these substances and loaded in the device. If water penetrates the polymeric device the loaded drug is then surrounded from the hydrophobic substances. These substances hinder the water from reaching the agent and keep the agent in a stable state.

After implantation, the initial dosage can be immediately released through an open channel. Following the administration schedule of the vaccines the material in the blocked channel will dissolve and the dosages are released. Fast pulsed release or slow release of the individual dosage can be controlled by the channel geometry. Alternatively, the initial dosages can be administrated directly during the injection procedure. Then, only booster dosages have to be loaded in the device. In another embodiment, the device can be loaded with up to 10 mg of active vaccines.

Preferred materials for the devices are polymers with a degradation time of 3-12 months, such as high molecular weight PLGA compounds. As a blocking polymer, fast degrading polymers or solvable substances, such as low molecular weight PLGA formulations or polyethylene glycol are preferred (e.g., degradation time of one month for the first booster dosage and 5 month for the second booster dosage). Methods for filling the channel include, but are not limited to, diverse printing, inkjet, dispensing technologies or methods based on capillary forces.

Examples of diseases that can require multiple dose vaccination include, but are not limited to, hepatitis A, hepatitis B, polio, mumps, measles, rubella, diphtheria, pertussis, tetanus, HiB, chicken pox, rotavirus, influenza, meningococcal disease and pneumonia. In order to provide best protection, children are recommended to receive vaccinations as soon as their immune systems are sufficiently developed to respond to particular vaccines, with additional booster shots often required to achieve full immunity. This has led to the development of complex vaccination schedules and routine vaccination of children. A device that can make the vaccination schedules easier to obtain and only requires minimal injections should make the vaccination process easier, especially in the case of children. Some embodiments of the device described herein may provide a better immunization method.

### Anchor Structures

One aspect of the invention incorporates an anchor structure into a delivery wing or the device body. Examples of the anchor structure on the delivery wing are, but are not limited to, jagged edges, hooks, needles, and pins. Other types of anchor structures can be incorporated into the body of the device including, but not limited to, holes, slits, and knobs for securing the device. Securing of the device can comprise adhering, grasping, clinging, stapling, suturing, or any combination thereof.

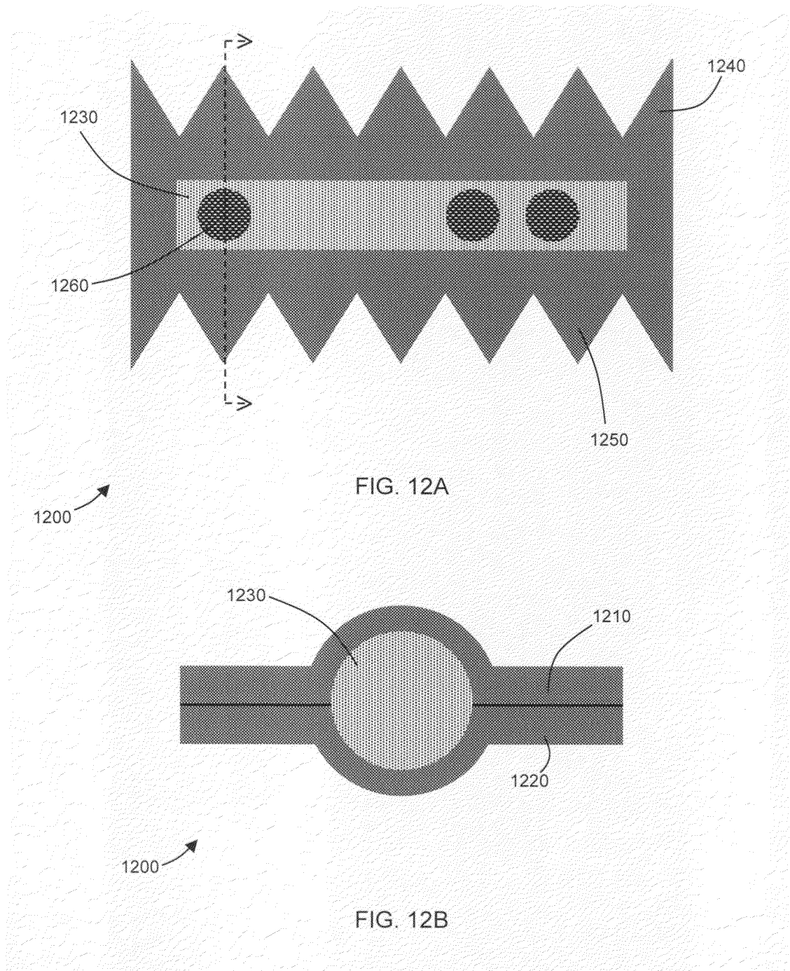

An embodiment of the invention comprising anchors is shown in FIG. 11, wherein anchors on the delivery wings 1130 are incorporated at one end of a device 1100 with a container 1110, delivery wings 1130, and multiple channels 1120. The anchor structure comprises a jagged structure 1140 at one end of the delivery wings 1130. Also, in this illustration, a hole 1150 is incorporated into the device body. Other examples incorporate one or more anchor structures in the body, at the end, or along the entire device.

Anchor structures have a variety of uses in delivering a therapeutic agent or anchoring a polymer sheet in place. Example treatments/diseases where it is useful for the device not to move include, but are not limited to, drug delivery to the eye, drug delivery to a target area, cancer chemotherapy, and dye or marker applications, such as radiation therapy seeds.

### Radiation Therapy

In another embodiment of the invention, anchor structures are useful for securing a device for radiation therapy imaging and/or delivery of therapeutic agents. Image-guided radiation therapy relies directly on the imaging modalities from planning as the reference coordinates for localizing the patient. The variety of image gathering hardware used in planning includes computed tomography (CT), magnetic resonance imaging (MRI), and positron emission tomography (PET) among others. Metal or alloy radiation markers, also referred to herein as seeds, are often implanted into a target tissue to provide a reference for image-guided radiation therapy. The typical size of the seeds ranges from sub-millimeters to millimeters to allow for administration with small devices, such as a needle or an endoscope.

The seeds can be made from materials such as gold or carbon, and can be shaped as rods or spheres. The seeds have good visibility with common radiation therapy imaging methods. A common problem with the implanted seeds is they migrate in the target tissue between radiation therapy doses. If the seeds migrate, the baseline image registration used to provide targeted image-guided radiation therapy can be inaccurate, potentially altering the treatment and treatment outcome. Conventional seeds often do not allow for differentiation after implantation.

FIG. 12A shows an embodiment of a radiation therapy guidance device 1200 and FIG. 12B shows a cross-sectional view of the device 1200 along the dashed lines in FIG. 12A. The device 1200 includes a seed 1260 located in a container 1230 embedded between polymer layers 1210, 1220. The seed 1260 can be, but is not limited to, a solid metal particle, such as a sphere, rod, or pressed powder. Using multiple seeds of different shapes or compositions generates a certain pattern for differentiation as well as for tracing after the device is implanted. It may be preferable to incorporate biodegradable polymer layers and anchors to avoid migration. Jagged line structures 1240, 1250 on the sides of the device 1200 are for anchoring the device 1200. Other anchoring structures, such as hooks, can also be incorporated.

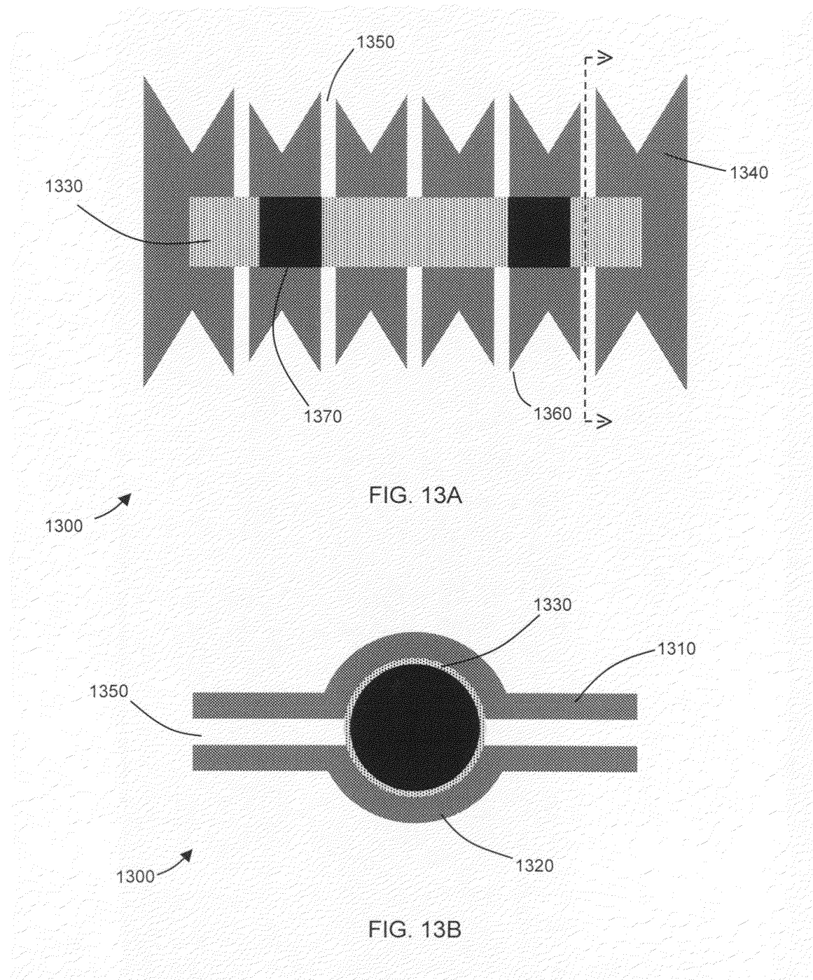

Furthering the scope of the invention, an embodiment can comprise a seed comprising a metal or alloy radiotherapy marker tube or film. FIGS. 13A-B demonstrate a device 1300 comprising an anchor structure 1360, a container 1330 (between polymer layers 1310, 1320), and delivery wings 1340 with channels 1350 to release an agent from the container 1330. In addition, the device comprises radiotherapy marker tubes 1370, allowing for travel of the agent within the container through the marker tube 1370. The radiotherapy marker tube 1370 or film comprises a thin or thick metal film pattern (coated inside or outside of the device 1300) incorporated into or onto the structure by means of standard thick or thin film technologies. As an example, the metal film width may vary between 0.1-10 micrometers. Agents in a container in this embodiment may be radio sensitizers, such as 5FU or gemcitabine for local delivery. The agents may also be chemotherapeutic agents for adjunct or adjuvant therapy with radiation treatment.

The device 1300 in FIGS. 13A-B, can also be combined with the device 1000 in FIG. 10 with delivery channels 1025, 1035 blocked by biodegradable polymers to control the release of radiosensitizing or chemotherapeutic agents. The degradation time of the material used to construct the delivery regions and containers can be optimized by polymer type in regards to a specific application. For example, the polymer can degrade in 2-3 months for devices containing seeds, according to standard radiation therapy treatment durations.

### Device Fabrication

Unlike existing devices with a two-dimensional bonded sheet or “three-dimensional” bonded planar layers, embodiments of the present invention involves creating a device with a large three-dimensional container for a high drug to polymer loading ratio and may include a delivery wing to space the timing and distance of agent release and delivery.

Fabrication methods are fully compatible to standard sheet fabrications techniques such as molding, forming, cutting, and extruding, as well as large sheet fabrication schema. In a preferred embodiment, the polymer layers are bonded together. The bonding technique preferably does not damage any pattern in a polymer layer. The bonding technique is preferably a low temperature method in cases where temperature sensitive inclusions are present. Suitable bonding methods include, but are not limited to, solvent bonding, solvent vapor bonding, flash thermal bonding, adhesive bonding, mechanical interlock bonding, plasma bonding or ultrasonic bonding. In some cases, it will be desirable to precisely register features of two (or more) polymer films being bonded. Known registration techniques (e.g., as in semiconductor lithography, mask aligning or wafer bonding techniques) are applicable.

Two bonding methods that are particularly useful for methods of fabrication are solvent bonding and flash thermal bonding. A solvent bonding process can be used to dissolve a thin sublayer (i.e., about 1 micron or less in thickness) of a first patterned polymer layer, then a second patterned polymer layer is placed in contact with the dissolved sublayer of the first polymer layer. Such a solvent bonding process can include liquid solvent spraying and/or solvent vapor condensation and/or exposure to a vapor solvent. A flash thermal process (e.g., using a non-contact infrared source) can be used to melt a thin sublayer (i.e., about 1 micron or less in thickness) of a first patterned polymer layer (without substantially affecting the remainder of the first layer) then a second patterned polymer layer is placed in contact with the melted sublayer of the first polymer layer. Both methods offer damage (and deformation) free bonding that preserve any patterned features in both layers.

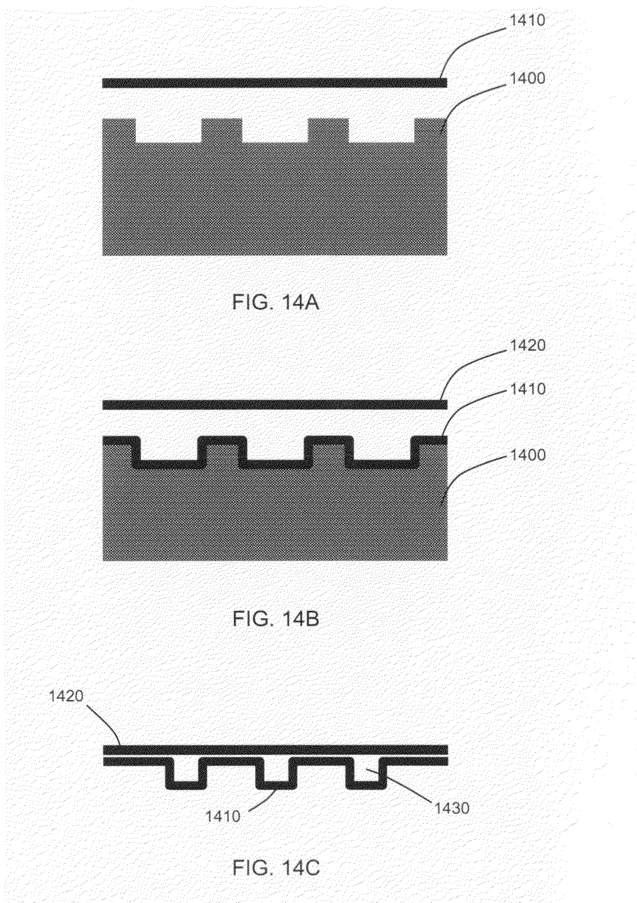

FIGS. 14A-C demonstrate an embodiment of a fabrication method involving fitting a first polymer layer 1410 to a mold 1400 and bonding a second polymer layer 1420 to the first polymer layer 1410, forming a three-dimensional container 1430. One or both of these polymer layers can comprise a biodegradable polymer. Conduits, such as channels, are incorporated into one of the polymer layers before the layers are bonded, or are incorporated after device fabrication. The method is an example of a fabrication schema that allows for post loading of an agent. Post loading methods of an agent into a container include, but are not limited to, an injection of an agent in solid, liquid, powder, or slurry formulation.

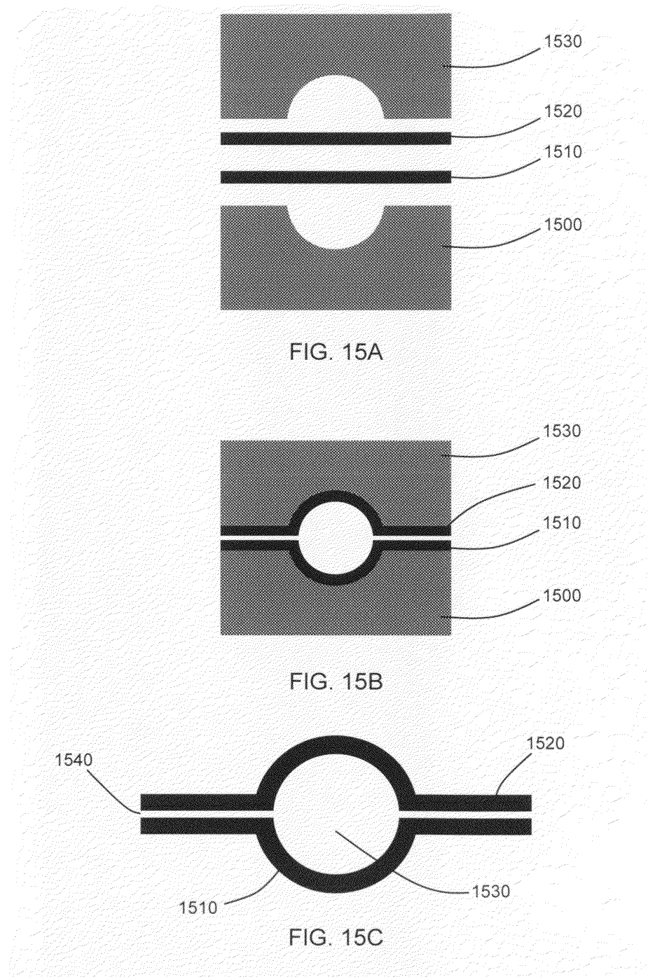

Another embodiment of a fabrication method is shown in FIGS. 15A-C, wherein a first polymer layer 1510 is shaped on a first mold 1500 and a second polymer layer 1520 is shaped on a second mold 1530. The two molded layers are then partially bonded together to form a large void in the non-bonded region comprising a three-dimensional container 1530 (e.g. cube, cone, sphere, or cylinder). Delivery channels 1540 are incorporated into one of the polymer layers before the layers are bonded, or are incorporated after device fabrication. The method can also be used to create a bonded delivery wing or region in the same process.

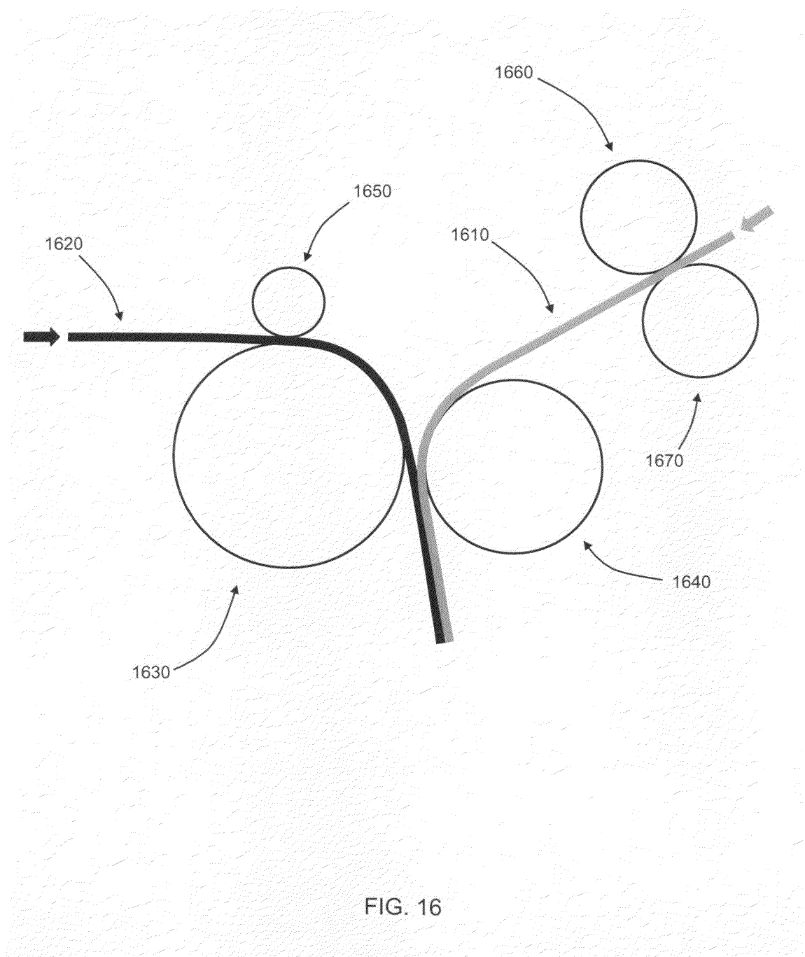

An example of a large sheet fabrication method is shown in FIG. 16, wherein individual layers are molded to a shape in a rolling process and then bonded together. The first layer 1610 is molded between a metal roll at about 150 C encompassing a mold 1660 for the delivery structures and a hard rubber roll 1670 at about 90 C. The second layer 1620 is molded to create the basis of the container between a soft rubber roll 1650 at about 120 C and a metal roll at about 50 C. encompassing the mold 1630. The two layers 1610, 1620 are then bonded together between the metal roll 1630 and another hard rubber roll 1640. The non-bonded areas formed in the molding process offer the voids comprising container and delivery conduits. Agents can be post loaded into a container. Post loading methods of an agent into a container include, but are not limited to, an injection of the agent in solid, liquid, powder, or slurry formulation.

### Therapeutic Applications

Embodiments and methods of the invention described herein are useful for a variety of therapeutic and non-therapeutic applications including, but not limited to, those diseases, treatments, and medical field mentioned herein. Some embodiments of the invention are useful for the treatment of pain, such as post-surgical pain, cancer pain, epidural injection, and nerve blocking. Examples of therapeutic agents that could be loaded into the devices described herein include bupivacaine and levo-bupivacaine.

Some embodiments include devices that can be implanted in an ocular region. Delivery to the eye of a therapeutic amount of an active agent can be difficult, if not impossible, especially for drugs with short plasma half-lives since the exposure of the drug to intraocular tissues is limited. A more efficient way of delivering a drug to treat an ocular condition is to place the drug directly in the eye. In one embodiment of the invention, the drug delivery device is sized and adapted for placement into an eye, for example into one of an anterior chamber of an eye and a posterior chamber of an eye or in the vicinity of the sclera. Examples of diseases that could benefit from therapeutic agents delivered by embodiments of the invention described herein include, but are not limited to, age-related macular degeneration and diabetic retinopathy. Examples of therapeutic agents useful for such treatments include corticosteroids, anti-VEGF, octreotide-sandostatin LAR, and 5-FU.

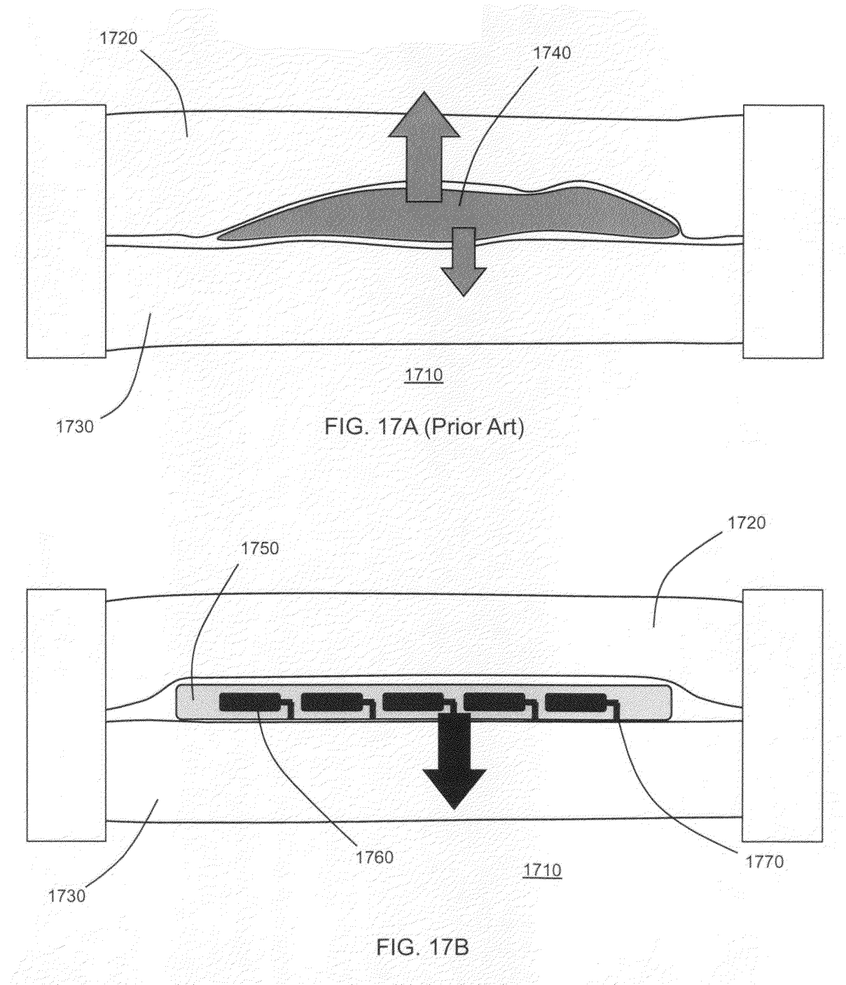

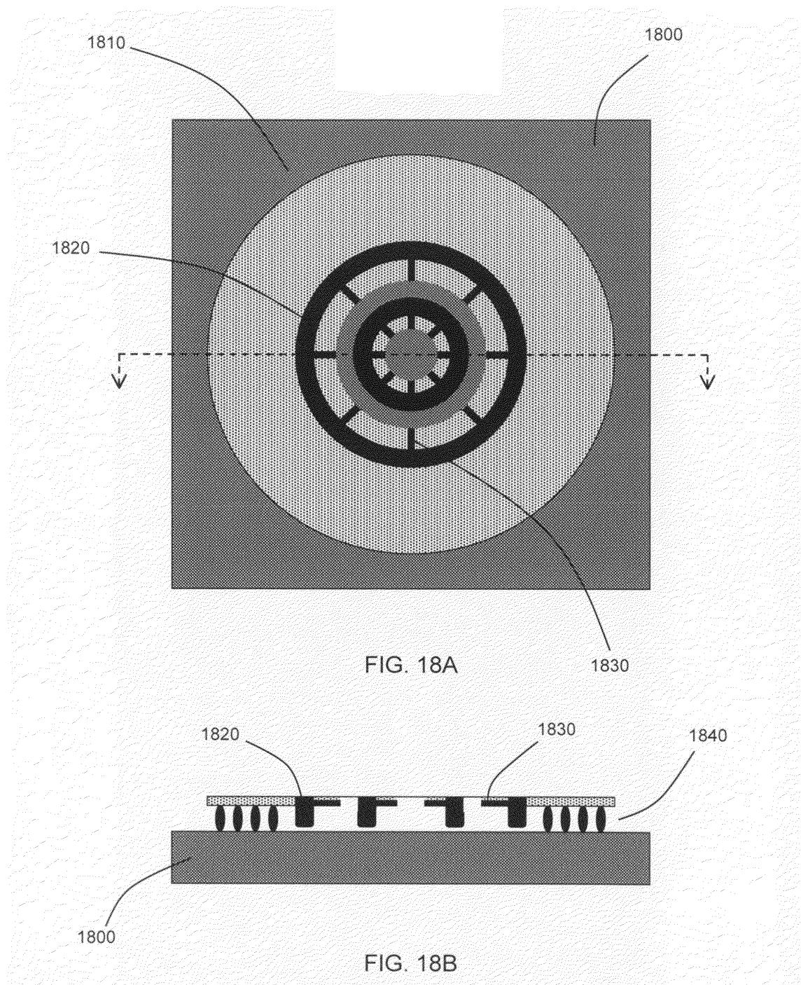

FIGS. 17B and 18A-B show embodiments of the present invention having advantages over existing treatments for the eye. It is noted that these embodiments are not restricted to treatments for the eye or sclera. FIG. 17A shows aqueous or gel depot in existing sclera treatment. The gel 1740 is located between the conjunctiva 1720 and the sclera 1730. However, in these existing treatment procedures, the drug delivery is non-directional, thereby allowing only less than 10% of the drug being delivered to the intra ocular region 1710. In addition, the drug is delivered in a burst at the beginning of the treatment and is not uniform in area. Furthermore, the loading ratio is typically very low with limited delivery duration.

FIG. 17B shows an embodiment of the present invention having a largely unidirectional delivery of drugs to the sclera 1730 with a high local drug concentration for efficient delivery. The device 1750 includes containers 1760 and channels 1770 that are open to only one side of the device 1750. In contrast to existing techniques, the precise rate and dosage can be determined by controlling the container and channel size and geometry. As described above, the loading ratio of drug to device material can be very high, greater than about 0.5 and up to about 0.9. In addition, the drug can be uniformly delivered over the treatment area and can provide long-term delivery. Lastly, the device 1750 can be implanted through non or minimally invasive office procedures.

FIG. 18A shows an embodiment of a thin sheet device 1810 for treating the sclera 1800. The disc-shaped device 1810 includes concentric ring-shaped containers 1820 and channels 1830 emanating from the containers. FIG. 18B shows a cross-sectional view of the device 1810 along the dashed lines in FIG. 18A. The cross-sectional view shows a further feature of sealing structures, such as micro rings 1840. The thickness of device 1810 can range between 100 μm to 2000 μm, or preferably between 100 μm to 300 μm. The embodiments of the present invention are not limited to disc-shaped devices, alternative shapes, such as rectangular-shaped devices, can also be used.

The device described herein is also useful for treating cancer. Examples of diseases and treatments include, but are not limited to, head and neck cancer, breast cancer after maxed radiation, ovarian cancer with abdominal spreading, advanced pancreas cancer, and recurrent sarcoma. Examples of therapeutic agents for treating cancer that could be loaded into the devices described herein include common chemotherapy agents and radiosensitizers.

Another therapeutic application of the device herein is for the field of orthopedics and includes treatments of spine fusion and deep venous thrombosis. Examples of therapeutic agents for treating orthopedic ailments include bone morphogenetic proteins and low molecular weight heparin.

Endocrinology is another medical field where the device described herein has a therapeutic benefit. Examples of diseases where therapy with the devices is useful include diabetes, obesity, and hormone delivery.

Another field that benefits from therapy from the devices is cosmetics. A number of agents, both therapeutic and cosmetic, are deliverable by the devices. Examples of such agents include botolinium toxin type A and other injectable cosmetics.

It will be appreciated by those skilled in the art that embodiments of the invention described herein are also useful for therapeutic and non-therapeutic uses for veterinary medicine. The devices are especially useful for delivery pain therapy to animals and other procedures that may require sutures. An injectable device that the animal would not harass would be useful in this instance.

Additional therapeutic and non-therapeutic applications wherein the device of the present invention can be used is described in U.S. patent application Ser. No. 11/402,651 filed Apr. 11, 2006, which is incorporated herein by reference in its entirety. U.S. patent application Ser. No. 11/402,651 also provides details regarding other agents that can be loaded in the devices of the present invention.

### Implantation

It will be appreciated that the device of the invention can be implanted using methods known in the art, including invasive, surgical, minimally invasive and non-surgical procedures. Depending on the subject, target sites, and agent(s) to be delivered the microfabrication techniques disclosed herein, can be adapted to make the delivery device of the invention of appropriate size and shape. The devices described herein are suitable for use in various locations in the body. For example, they can be implanted on the surface of the skin, under the skin, or in or near internal tissues or organs.

### Animal Studies

In vitro and in vivo experiments using example devices of the present invention were performed on test rabbits. For the in vivo study, three devices were implanted in each test rabbit to study the wound healing of the incision site and drug release of the devices. Each device held 1.5 mg HCl bupivacaine (pure) to be released to the rabbit. The devices were implanted subcutaneously using a needle device applicator in minimally invasive implantation procedures. Each device was 12 mm by 3 mm and contained 500 microchannels, each microchannel having a length of 1 mm. The release rate of the bupivacaine was 50 μg/day. Results of the in vivo experiments found no or minimal signs of infection, inflammation, edema, or erythema at the incision sites. These results indicate excellent biocompatibility and device robustness.

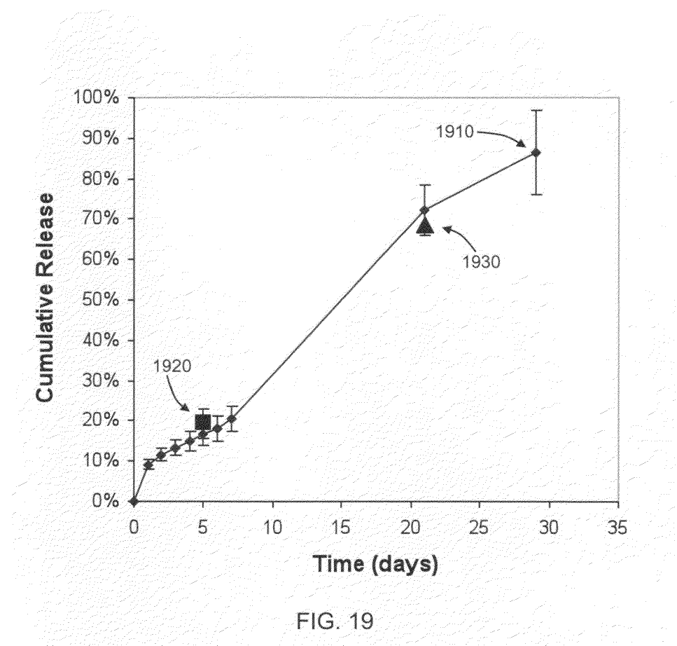

The drug release data was also recorded in the experimental studies. In particular, device explantation was performed to study the devices and tissue samples. Explantation of a first group of rabbits was performed after 5 days of device implantation and explantation of a second group of rabbits was performed after 21 days of device implantation. FIG. 19 shows a plot of cumulative drug release versus time for these two groups from the in vivo study and a comparison with in vitro studies. The cumulative drug release is based on residual drug amounts in the device after explantation. The connected diamonds 1910 indicate the in vitro release measurements over time. The square 1920 represents the first explantation group and the triangle 1930 represents the second explantation group from the in vivo studies. As can be seen in the plot, the in vivo release showed a good correlation with the in vitro data. Furthermore, a large percentage of drug release was seen for the in vivo data demonstrating effective therapy treatment. In particular, after 21 days, at least 65% of the drug was released from the devices in the in vivo study.

As one of ordinary skill in the art will appreciate, various changes, substitutions, and alterations could be made or otherwise implemented without departing from the principles of the present invention, e.g. any number and geometry of containers and channels can be used or several embodiments of the invention described herein can be combined to comprise additional embodiments of the invention. Accordingly, the scope of the invention should be determined by the following claims and their legal equivalents.

What is claimed is:

1. A device for releasing an agent to a subject, said device comprising:
a first polymer layer and a second polymer layer, wherein said first and said second polymer layers are partially bonded to form a bonded region and a non-bonded region,
wherein said non-bonded region forms at least one container located between said first and said second polymer layers, wherein said container holds said agent to be released to said subject,
wherein said bonded region comprises a delivery region, wherein said delivery region comprises at least one channel located between said first and said second polymer layers, wherein said channel connects said container to an external environment of said device, and wherein said channel allows said agent to be released to said subject.

2. The device as set forth in claim 1, wherein said device has a high loading ratio of said agent to polymer material of said polymer layers, and wherein said high loading ratio is greater than about 0.5.

3. The device as set forth in claim 1, wherein said polymer layers are biodegradable or biocompatible, and wherein said agent is a therapeutic agent for providing therapy to said subject.

4. The device as set forth in claim 3, wherein said polymer layers are biodegradable, and wherein said biodegradable polymer layers have an in vivo lifetime greater than the duration of the therapy.

5. The device as set forth in claim 3, further comprising a biodegradable material in said channel, wherein said channel is at least partially blocked by said biodegradable material, wherein said polymer layers are biodegradable, and wherein the in vivo lifetime of said biodegradable material in said channel is less than the in vivo lifetime of said biodegradable polymer layers.

6. The device as set forth in claim 1, wherein said container has a thickness ranging from about 0.01 mm to about 3 mm.

7. The device as set forth in claim 1, wherein said at least one delivery region comprises at least one delivery wing, and wherein said at least one delivery wing is flexible.

8. The device as set forth in claim 7, wherein said at least one delivery wing can be folded or rolled to fit inside of a hollow instrument, and wherein said hollow instrument is for inserting said device into said subject.

9. The device as set forth in claim 8, wherein said hollow instrument is selected from a group consisting of a needle, a catheter, an endoscope, and a syringe.

10. The device as set forth in claim 8, wherein said flexible delivery wing unfolds after insertion into said subject.

11. The device as set forth in claim 1, wherein said non-bonded region comprises a plurality of containers, and wherein said delivery region in said bonded region comprises one or more channels.

12. The device as set forth in claim 11, wherein said delivery comprises at least two channels corresponding to two different containers, wherein each of said at least two channels connects said corresponding container to said external environment of said device, wherein said at least two channels have different geometries, and whereby said different geometries provide different delivery rates of said therapeutic agent from said corresponding containers.

13. The device as set forth in claim 11, further comprising a biodegradable material in one of said channels, wherein the same of said channels is at least partially blocked by said biodegradable material, whereby said biodegradable material provides delayed release of said agent in said container connected to the same of said channels.

14. The device as set forth in claim 11, further comprising a first biodegradable material in one of said channels, referred to as a first channel, and a second biodegradable material in another of said channels, referred to as a second channel, wherein said first channel is at least partially blocked by said first biodegradable material, wherein said second channel is at least partially blocked by said second biodegradable material, and wherein the in vivo lifetime of said first biodegradable material is different than the in vivo lifetime of said second biodegradable material.

15. The device as set forth in claim 11, further comprising two or more different therapeutic agents, wherein said different therapeutic agents are stored in separate of said containers.

16. The device as set forth in claim 11, wherein said device has a sheet structure for releasing said agent over a large area of said subject.

17. The device as set forth in claim 11, wherein said device has a tubular shape.

18. The device as set forth in claim 11, further comprising a mixing channel, wherein said mixing channel connects one of said containers to another of said containers.

19. The device as set forth in claim 1, further comprising an anchor for maintaining a position of said device.

20. The device as set forth in claim 19, wherein said anchor comprises a jagged structure, a suture opening, a hook, a needle, a pin, a hole, a slit, a knob, or any combination thereof.

21. The device as set forth in claim 19, wherein said device is implantable in an eye of said subject.

22. The device as set forth in claim 1, wherein said at least one channel of said delivery region releases said agent in approximately one direction.

23. A method for releasing an agent to a subject, said method comprising:
(a) having an agent-delivery device, wherein said device comprises:
a first polymer layer and a second polymer layer, wherein said first and said second polymer layers are partially bonded to form a bonded region and a non-bonded region,
wherein said non-bonded region forms at least one container located between said first and said second polymer layers, wherein said container holds said agent to be released to said subject,
wherein said bonded region comprises a delivery wing, wherein said at least one delivery wing is flexible, wherein said delivery wing comprises at least one channel located between said first and said second polymer layers, wherein said channel connects said container to an external environment of said device, and wherein said channel allows said agent to be released to said subject;
(b) loading said container of said agent-delivery device with said agent;
(c) folding said at least one delivery wing;
(d) positioning said agent-delivery device with said folded delivery wing into a hollow instrument; and
(e) injecting said agent-delivery device into said subject from said hollow instrument, whereby said agent is released to said subject.

24. The method as set forth in claim 23, wherein said agent-delivery device has a high loading ratio of said agent to polymer material of said polymer layers, and wherein said high loading ratio is greater than about 0.5.

25. The method as set forth in claim 23, further comprising at least partially filling said channel with a biodegradable material, wherein said polymer layers are biodegradable, and wherein the in vivo lifetime of said biodegradable material in said channel is less than the in vivo lifetime of said biodegradable layers.

26. The method as set forth in claim 23, wherein said agent is released in approximately one direction from said device.

27. The method as set forth in claim 23, further comprising anchoring said agent-delivery device to said subject.

28. The method as set forth in claim 27, wherein said anchoring comprises adhering, grasping, clinging, stapling, suturing or any combination thereof.

29. A radiation therapy guidance device, said device comprising:
a first polymer layer and a second polymer layer, wherein said first and said second polymer layers are partially bonded to form a bonded region and a non-bonded region, and wherein said non-bonded region forms at least one container located between said first and said second polymer layers; and
a radiation therapy imaging marker, wherein said radiation therapy imaging marker is stored inside of said container or is attached on the outside of said container.

30. The device as set forth in claim 29, further comprising an anchor for maintaining a position of said radiation therapy guidance device.

31. The device as set forth in claim 30, wherein said anchor comprises a jagged structure, a suture opening, a hook, a needle, a pin, a hole, a slit, a knob, or any combination thereof.

32. The device as set forth in claim 29, wherein said radiation therapy imaging marker comprises one or more solid particles, and wherein said solid particles comprise metal, metal alloy, gold, carbon, or any combination thereof.

33. The device as set forth in claim 29, wherein said radiation therapy imaging marker comprises a metal film, and wherein said metal film is coated on said radiation therapy guidance device.

34. The device as set forth in claim 29, wherein said container holds an agent, wherein said bonded region comprises at least one channel, wherein said channel connects said container to an external environment of said device, and wherein said channel allows said agent to be released to said external environment.

35. The device as set forth in claim 29, wherein said polymer layers are biodegradable or biocompatible.
